(19) United States
(12) Patent Application Publication
(10) Pub. No.: US 2006/0292248 A1
(43) Pub. Date: Dec. 28, 2006

(54) Biological attack protection kit and method

(75) Inventor: Robert Heiman, Mission Viejo, CA (US)

Correspondence Address: CONNORS ASSOCIATES, 1600 DOVE ST, SUITE 220, NEWPORT BEACH, CA, 92660, US

(73) Assignee: Bio-Germ Protection, LLC

(21) Appl. No.: 11/156,692

(22) Filed: Jun. 20, 2005

(51) Int. Cl. A61K 36/752; A61K 35/64; A61M 11/00

(52) U.S. Cl. 424/736; 128/200.23; 424/769; 424/539

(57) ABSTRACT

A portable kit includes one or more different forms of bio-attack protection agents that are effective in killing or disabling harmful biological substances. The kit is easy to transport and is practical for normal, every day use. It contains one or more vessels holding bio-attack protection agents thereby providing personal protection to individuals in case of a biological attack.

The inventor incorporates herein by reference any and all U.S. patents, U.S. patent applications, and other documents, hard copy or electronic, cited or referred to in this application.

### Definitions

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

“Bio-attack protection agent(s)” shall mean any means that renders a harmful biological substance incapable of causing injury or reducing or eliminating the deleterious effects of a harmful biological substance.

“Rectangular” includes square.

### BACKGROUND OF INVENTION

The threat of a biological attack is of great and immediate concern. Because there is no way of predicting when such an attack may arise, to provide personal protection in case of an attack presents a major challenge. For example, anthrax has been used already in such an attack. Certain bio-attack protection agents to counter such an anthrax attack are discussed in The Journal of Burn & Wounds, entitled “Combating Anthrax,” Dec. 6, 2004.

### SUMMARY OF INVENTION

In order for personal protection to be truly effective, the means for combating the harmful biological substance must be portable. The present invention provides a portable kit that is easy to transport and a method for combating a biological attack. The kit and method of this invention are particularly useful in protecting against an attack where a harmful biological substance is dispersed in the atmosphere, for example, anthrax or other harmful biological substance. The kit includes one or more vessels holding a bio-attack protection agent. Optionally, the kit also includes a facemask that filters from air harmful, airborne biological substances.

After reading the following section entitled “DETAILED DESCRIPTION OF SOME EMBODIMENTS OF THIS INVENTION,” one will understand how the features of this invention provide its benefits. The benefits of this invention include, but are not limited to: (a) allowing individuals to carry with them personal protection against one or more different types of biological threats, (b) providing individuals with a sense of security knowing that in the event of an unexpected biological attack they have the necessary items to protect themselves, (c) providing one or more defenses against a biological attack in a kit that is small enough and light enough that it is practical for the user to keep it with them essentially at all times.

Without limiting the scope of this invention as expressed by the claims that follow, some, but not necessarily all, of its features are:

The kit of this invention may store one or more vessels holding a bio-attack protection agent or agents. In one embodiment, the kit includes a manually actuated aerosol spray container holding a bio-attack protection agent, a manually actuated inhaler containing a bio-attack protection agent to be sprayed into the atmosphere, and a receptacle of topical skin lotion including a bio-attack protection agent. The aerosol spray container may contain just enough aerosol spray to treat an interior of a sealed enclosed space having an approximate maximum volume of about 6,000 cubic feet. This may be about 1.5 ounces of the aerosol spray. The inhaler may contain just enough of the bio-attack protection agent to allow the user to take at least 30 deep breaths at 10-minute intervals. This may be approximately 1.0 ounces of the bio-attack protection agent. The inhaler may be a flexible, resilient bottle or the like vessel constructed so that the user may manually squeeze the inhaler to expel a mist that may be directly inhaled through a nostril of the user. The skin lotion receptacle may be sized such that it holds enough lotion to allow the user to cover substantially his or her entire body 2 to 3 times. This may be approximately 2.0 ounces of the lotion. The receptacle may have a re-sealable cover that allows the user to apply a portion of the lotion and then re-seal the receptacle. Optionally, a facemask may be included in the kit. While worn, the facemask filters inhaled air to remove harmful airborne biological substances. The mask is sized and shaped to fit comfortably over the user's nose and mouth, thus allowing the user to breathe naturally while the mask filters out harmful biological substances. Having a plurality of different forms of protection means a single kit could enable the user to select the most appropriate form to counter a biological attack. The effectiveness of the invention, however, is maximized by using all products in the sequential order as discussed subsequently.

There are other features of the kit. It may have a case in which the vessels are stored. In one embodiment it looks like a brief case and may include a handle for ease of transportation and may also include at least one locking device to keep the kit in a closed position. It is desirable for the kit to be compact to minimize its volume. For example, it may have an overall volume below about 144 cubic inches. The case may form an enclosure that is opened manually to access the vessel or vessels holding the bio-attack protection agent or agents. The enclosure may have an open recess in which may be seated a liner that may have one or more cavities therein that retain the removable vessels stored therein. The case may be made of a durable, rigid material to help protect against damage by an occasional impact or the normal wear and tear of every-day travel. Optionally, the kit may be packaged or otherwise designed to float on water. For example, the kit may be enclosed and sealed within a transparent envelope or bag having pockets inflated with air. The bag may serve a multitude of functions including, but not limited to, assuring the user that the vessels in the kit have not been tampered with prior to sale, assuring the user that the contents of the kit have not been tampered with prior to use, and protecting the contents of the kit from the weather.

These features are not listed in any rank order nor is this list intended to be exhaustive.

A variety of different bio-attack protection agents may be used in this invention. It is desirable that a major portion of any bio-attack protection agent be a naturally occurring composition or compositions. The contents of the spray container may comprise a liquid blend of citrus oils, plant herbs, and seed bitters in a water and food grain alcohol solution. For example, the bio-attack protection agent in the spray container may include one or more of the following: bee propolis extract, melaleuca alternifolia (tea tree) leaf oil, urea, citrus aurantium amara (bitter orange/petitgrain) oil, citrus grandis (grapefruit) fruit extract. The contents of the inhaler may comprise a liquid blend of citrus oils, plant herbs, and seed bitters in an aqueous solution. For example, the bio-attack protection agent in the inhaler may include one or more of the following: citrus grandis (grapefruit) extract, alternifolia (tea tree) leaf oil. The contents of the skin lotion receptacle may comprise a liquid blend of citrus oils, plant herbs, and seed bitters in a base of olea europaea (olive) fruit oil. For example, the bio-attack protection agent in the skin lotion may include one or more of the following: bee propolis extract, urea, alternifolia (tea tree) oil, citrus grandis (grapefruit) seed, and fruit extracts.

This invention also includes a method of protecting an individual from a biological attack. One embodiment of this method comprises identifying what harmful biological substance potentially is to be used in the attack, and providing one or more bio-attack protection agents in a portable kit that enables a user to travel with the kit and have access to such bio-attack protection agents during an attack. Frequently, users will be traveling in an automotive vehicle. The kit is carried in the vehicle and it includes the appropriate bio-attack protection agents to counter an attack by the identified harmful biological substance. If the attack includes dispersing the harmful biological substance is the atmosphere, the kit will include at least the manually actuated aerosol spray container holding a bio-attack protection agent. During such an attack, while within the interior of the vehicle (and the vehicle preferably stopped), and with the interior of the vehicle providing an enclosed space substantially isolated from ambient air outside this enclosed space, the user sprays at least some of the bio-attack protection agent in the spray container into the vehicle's enclosed space.

One embodiment of the method of this invention specifically counters a biological attack in which a harmful biological substance is dispersed in the atmosphere. This method comprises the steps of

(a) providing a user with a portable kit comprising a manually actuated aerosol spray container holding a bio-attack protection agent, a manually actuated inhaler containing a bio-attack protection agent, and a receptacle of skin lotion including a bio-attack protection agent, and

(b) in an enclosed space substantially isolated from ambient air outside the enclosed space, while within the enclosed space, the user may in sequence,
(i) sprays at least some of the bio-attack protection agent in the spray container into the enclosed space,
(ii) inhales at least some of the bio-attack protection agent in the inhaler, and
(iii) applies at least some of the some of the lotion to exposed skin areas.
   The user may lastly cover his or her nose and mouth with a facemask that filters inhaled air to remove substantially harmful airborne biological substances. When the biological substance is anthrax, the bio-attack protection agent kills or inhibits the deleterious effects of anthrax. The bio-attack protection agents discussed above are useful for combating an anthrax attack.

### DESCRIPTION OF THE DRAWING

Some embodiments of this invention, illustrating all its features, will now be discussed in detail. These embodiments depict the novel and non-obvious biological attack protection kit and method of this invention as shown in the accompanying drawing, which is for illustrative purposes only. This drawing includes the following figures (Figs.), with like numerals indicating like parts:

FIG. 1 is a perspective view of one embodiment of a biological attack protection kit of the present invention enclosed within a transparent protective storage bag.

FIG. 2 is a perspective view of the biological attack protection kit depicted in FIG. 1 but with the protective storage bag removed.

FIG. 3A is a perspective view of the biological attack protection kit depicted in FIG. 2 but with the kit's case in an open position, revealing different means of protection from harmful biological substances.

FIG. 3B is a perspective view of the biological attack protection kit depicted in FIG. 3A but with the four different means of protection removed from the kit.

FIG. 3C is a perspective view of the biological attack protection kit depicted in FIG. 3A but with the internal lining removed.

FIG. 4 is a perspective view of an alternate embodiment of the biological attack protection kit of this invention showing the kit opened.

FIGS. 5 through 8 illustrate the method of this invention where:

FIG. 5 shows an aerosol spray being used to treat the air of a sealed room or enclosed space in an automotive vehicle.

FIG. 6 shows an inhaler being inserted into a nostril of the user prior to inhalation.

FIG. 7 shows a lotion being applied to a user's skin.

FIG. 8 is a side view of a facemask attached to the user's head.

### DETAILED DESCRIPTION OF SOME EMBODIMENTS OF THIS INVENTION

General

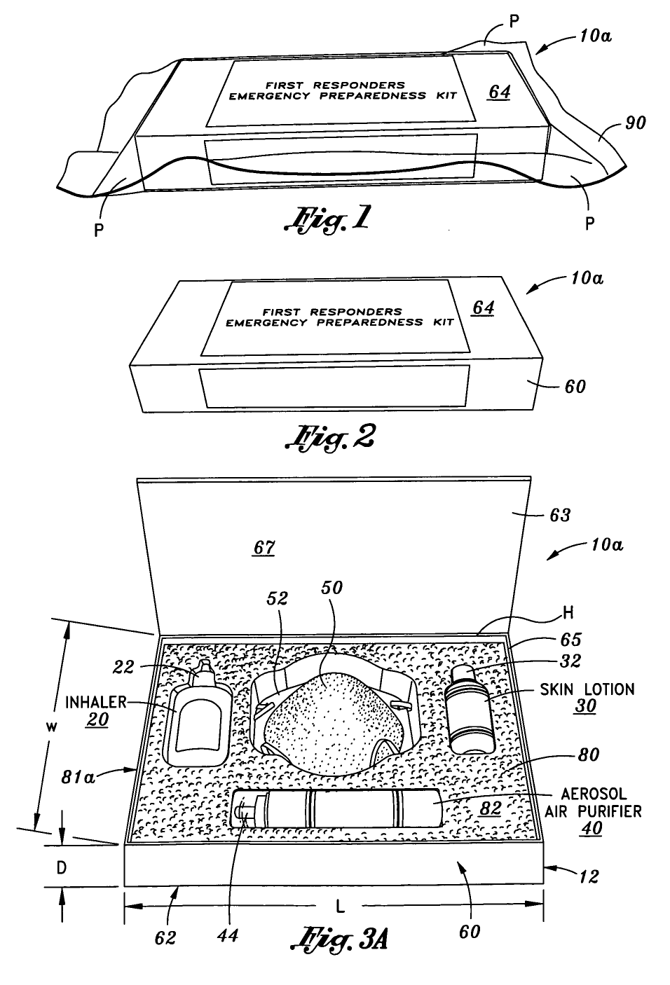

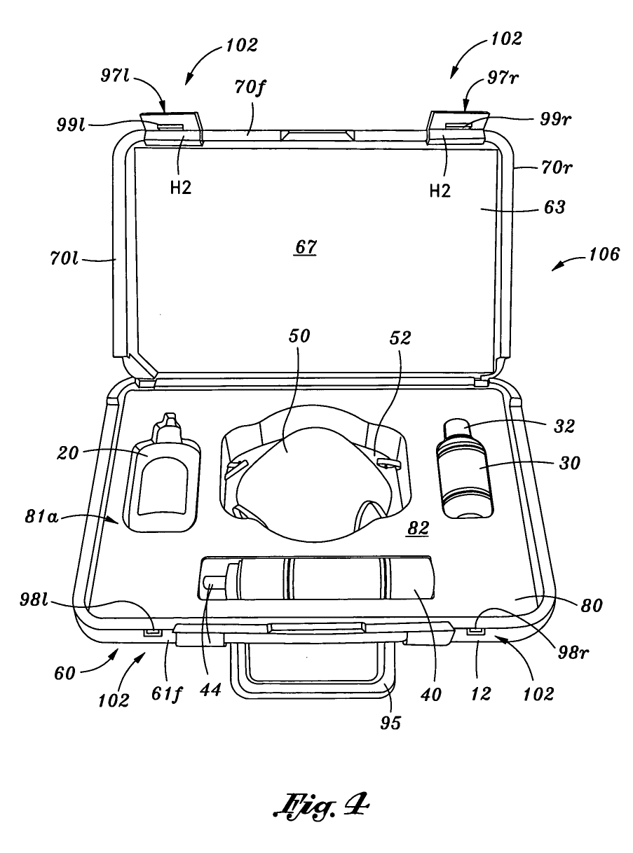

This invention comprises a method for countering a biological attack and a portable kit containing bio-attack protection agents to be used in the event of an attack. FIGS. 1 through 3C depicted one embodiment of the kit of this invention that is designed to float on water. FIG. 4 depicts an alternate embodiment of the kit that is easy to carry like a brief case. FIGS. 5 through 8 illustrate the method of this invention.

Both embodiments of the kit, the kit 10a (FIGS. 1 through 3C) and the kit 10b (FIG. 4) include four different forms of protection. Three of these forms necessitate their own individual storage and application vessels, namely, an aerosol spray container 40 holding a bio-attack protection agent, a manually actuated inhaler 20 holding a bio-attack protection agent, and a receptacle 30 holding a skin lotion including a bio-attack protection agent. The various bio-attack protection agents are discussed above. The fourth, a conventional filter-type facemask 50, is itself a form of protection, and is optional.

FIGS. 1 through 3C and FIG. 4

Each kit 10a and 10b is portable to enable a user to carry the kit with him or her essentially at all times. Each kit 10a and 10b includes a case 60 made of a rigid material capable of withstanding impacts, poking, or other abuses that may befall an object of its size traveling with the user. The case 60 includes a base 12 and a lid 63 that when closed, protects the kits' contents and, when opened, allows the user to have easy access to the protection means stored within the kits.

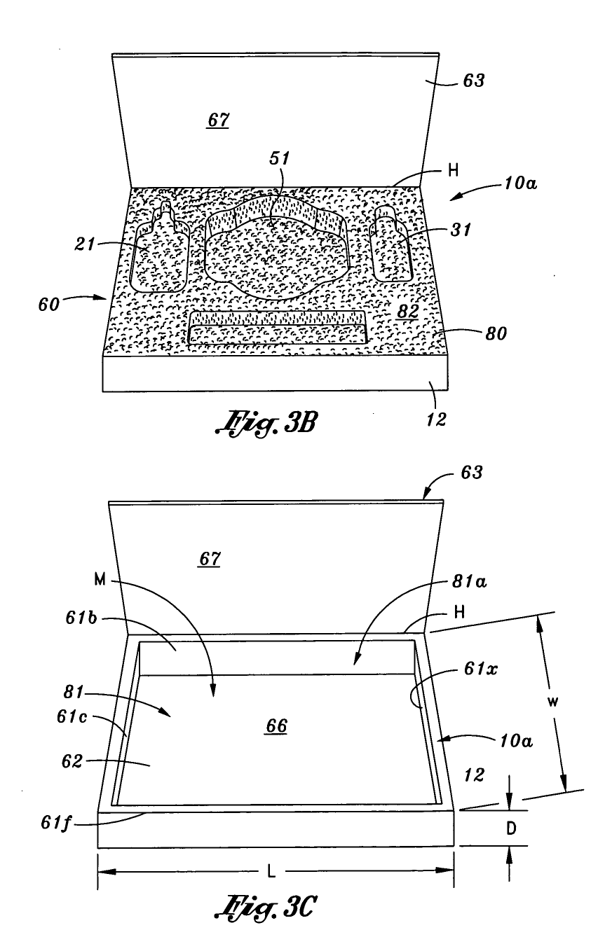

As best shown in FIG. 3C, the base 12 is substantially box shaped and may have, for example, a length L from about 12 to about 15 inches, and a width W from about 6 to about 8 inches, and a depth D from about 2 to about 3 inches. The depth D of the base 12 is greater than the thickness of any of the storage vessels for the bio-attack protection agents, or any other device that may be included in the kit 10. The base 12 includes an open recess 81 created by a left sidewall 611, a right sidewall 61r, a front sidewall 61f, a back sidewall 61b that are normal to each other and are perpendicular to a bottom wall 62, which is a substantially rectangular, flat sheet. The open recess 81 has an open, rectangular mouth M that the lid 63 covers. The lid 63 may be detached, or as illustrated, connected by a living hinge H along a top edge of, for example, the back sidewall 61b. The lid 63 is a substantially rectangular, flat sheet with dimensions slightly less than the dimensions of the open mouth M so that, when the lid is in the closed position (FIG. 2), it fits within the open mouth M, thus making the lid's top surface 64 and the top edges of the sidewalls 611, 61r, and 61f substantially flush with one another. The left sidewall 611 and right sidewall 61r are substantially parallel to each other, and the front sidewall 61f and back sidewall 61b are substantially parallel to each other. The upper edges of the sidewalls 611, 61r, 61f, and 61b define the outer perimeter of the open mouth M of the recess 81, and a top surface 66 (FIG. 3C) of the base bottom wall 62 defines the bottom of the recess 81.

As best seen in FIGS. 3A and 4, the aerosol spray container 40, the inhaler 20, the skin lotion receptacle 30, and the facemask 50 are securely held in place in the kits 10a and 10b by an internal lining 80 that fits snugly within and substantially fills the open recess 81. The internal lining 80 is made of, for example, polyurethane foam, and it has length and width dimensions substantially the same as the internal dimensions of the open recess 81. The lining 80 has a height, however, that is less than the depth of the open recess 81, with the difference between the height of the lining 80 and the depth of the open recess 81 being greater than the thickness of the lid 63 to provide space between a top surface 82 of the lining 80 and the inside surface 67 of the lid 63 when it is closed.

As best illustrated in FIGS. 3A and 3B, the lining 80 has a cavity 21 that is shaped to correspond to the shape of the inhaler 20, a cavity 31 that is shaped to correspond to the shape of skin lotion receptacle 30, a cavity 41 that is shaped to correspond to the shape of the aerosol spray container 40, and a cavity 51 that is shaped to correspond to the shape of the facemask 50. Thus, the cavities substantially match the shapes of the individual protection devices to be lodged therein. In other words, the shape of each cavity is substantially similar to the shape of the item it is intended to hold. This helps the user know where to return the inhaler 20, receptacle 30, container 40, or facemask 50 after using it, and helps to identify if one of these particular forms of protection is missing from the kit 10a or 10b.

As shown in FIG. 3B, the cavity 21 has a perimeter and depth that enables the inhaler 20 to be inserted into this cavity 21 and lay flat therein. The depth of the cavity 21 is more than the thickness of the inhaler 20 so that the inhaler 20 can lie completely below the top surface 82 of the lining 80 when inserted therein. The cavity 31 has a perimeter and depth that enables the skin lotion receptacle 30 to be inserted into this cavity 31. The depth of the cavity 31 is about half the diameter of the receptacle 30 so that the top surface 82 of the lining 80 comes to about the mid-point of the receptacle 30 when inserted therein. The cavity 41 has a perimeter and depth that enables the aerosol container 40 to be inserted into this cavity 41. The depth of the cavity 41 is about half the diameter of the aerosol container 40 so that the top surface 82 of the lining 80 comes to about the mid-point of the aerosol container 40 when inserted therein. The facemask cavity 51 has a perimeter substantially similar to the border 52 of the facemask 50. To avoid crushing the facemask 50, the depth of the cavity 51 is greater than the height of the facemask 50 so that the top surface 82 of the lining 80 is above the top most point of the facemask 50 when inserted therein. Any portions of the skin lotion receptacle 30 and aerosol container 40 extending above the surface 82 of the lining 80 are within the space between the top surface 82 and the inside surface 67 of the lid 63.

FIG. 1 shows the kit 10a enclosed within an airtight transparent plastic bag 90 that helps to protect and preserve the protection means stored within the kit 10a. The kit 10a may be sold enclosed within the bag 90. The bag 90 may remain on the kit 10a until the user needs to use the kit 10a. The bag 90 serves multiple functions including, but not limited to, (1) assuring the consumer that the protection means of the kit 10a have not been tampered with prior to, or after the initial sale and prior to the first use, and (2) protecting the protection means of the kit from the weather. Additionally, the bag 90 may have inflated pockets P that make the combination of the kit 10a wrapped in the bag 90 buoyant. This allows the kit 10a to float on water, should the kit 10a be used on a ship, or otherwise, where it might be dropped into water.

The kit 10b depicted in FIG. 4 is substantially similar to the kit 10a depicted in FIGS. 1 through 3C with the only exception being that its case 60b has a few additional features: a handle 95 and a locking device 102. The handle 95 is a substantially U-shaped handle 95 and it extends outward from the base 12 of the kit 10b. The handle 95, which is centered along the base front sidewall 61f, enables the user easily to carry the kit 10b with one hand. A left handle base 961 and a right handle base 96r located along the front sidewall 61f receive, respectively, opposed handle ends extending from the left and right sides of the handle 95 to attach the handle to the base 12.

The locking device 102 keeps the lid 63 closed until manually opened by a user. This locking device 102 includes a pair of clasps 971 and 97r each mounted by a hinge H2 along a front edge 70f of the lid 63. The left clasp 971 is located on the left side of the front edge 70f, with its left most edge being inward from the left edge 701 of the lid 63. The right clasp 97r is identical in size and shape to the left clasp 971, the only difference being that it is situated on the right side of the front edge 70f of the lid 63. The right most edge of the clasp 97r is inward from the right edge 70r of the lid 63. The inward displacement from their respective adjacent lid edges is the same distance. Within the center of both the left 971 and right 97r clasps is a substantially rectangular shape opening 991 and 99r, respectively. Two catches 981 and 98r on the front sidewall 61f of the kit base 12 are in alignment with the clasps 971 and 97r. Both catches 981 and 98r are identical in shape and each are designed to pass through the respective openings 991 and 99r and remain inserted therein upon closing of the lid 63, holding the lid closed, until the user manually disengages the catches 981 and 98r from the opening 991 and 99r. Because two clasps 971 and 97r are used to hold the kit 10b in a closed position, there is twice the security, since both clasps would have to fail, or be accidentally released, for the protection means within the kit 10b to be exposed and removed.

During a biological attack the user accesses the protection means within the kit 10a or 10b. To open kit 10a (FIG. 1), the user first removes the plastic bag 90 from around kit 10a and then opens the lid 63. This step is unnecessary with the kit 10b, since the user only needs to unlock the clasps 971 and 97r and open the lid 63. Opening the lid 63 exposes the protection means within the kits 10a and 10b, enabling the user to access these protection means. In the event that the attack disperses a harmful biological substance in the atmosphere, the user then uses in accordance with the method of this invention for his or her individual protection a bio-attack protection agent in an enclosed space substantially isolated from ambient air outside the enclosed space as depicted in FIGS. 5 through 8.

FIGS. 5 through 8

FIGS. 5 through 8 illustrate the method of this invention for combating a biological attack in which a harmful biological substance is used. One bio-attack protection agent may be used that is effective against a wide variety of different harmful biological substances, or a kit similar to those discussed above may include a plurality of vessels, each holding one type of bio-attack protection agent particularly suited to combat a specific type of harmful biological substance. When the user identifies, or is informed, for example over a radio broadcast, of the specific type of harmful biological substance he or she is facing, the user opens the kit to access the appropriate protection means. For example, the user selects and uses the one vessel containing the bio attack protection agent that is most effective against the specific type of harmful biological substance employed in the attack. In general, at least one of the vessels is an aerosol container holding this most effective bio-attack protection agent.

In the event that the harmful biological substance is airborne, the user enters, if not already within, an enclosed space substantially isolated from ambient air outside the enclosed space. This enclosed space may be a room or an automotive vehicle. Although bio-attack protection agent may be used in a different sequence within the enclosed space, the following sequence is preferred: (1) first spray a bio-attack protection agent into the enclosed space, (2) next inhale a bio-attack protection agent, and (3) thirdly apply to exposed skin areas a lotion including a bio-attack protection agent. Optionally, the user lastly covers his or her nose and mouth with a facemask that filters inhaled air to remove harmful, airborne, biological substances.

Referring to the kits 10a and 10b:

FIG. 5 shows an individual using the bio-attack protection agent in the aerosol container 40. As best he or she can, the user seals off from the ambient air outside an enclosed space 100 in which he or she is sheltered. Once the enclosed space 100 is sealed off, the user removes the aerosol container 40 and depress an actuator 44, spraying at least some of the contents of the aerosol container 40 into the atmosphere in the enclosed space to kill or otherwise ameliorate whatever harmful, airborne biological substances may be present in the enclosed space 100. The larger the volume of the enclosed space 100, the greater will be the amount of the bio-attack protection agent needed to eliminate effectively the biological threat. Once the user has finished with the aerosol container 40, he or she may return the aerosol container to the cavity 41 in the kit 10a or 10b, or discard the aerosol container if the contents therein have been depleted.

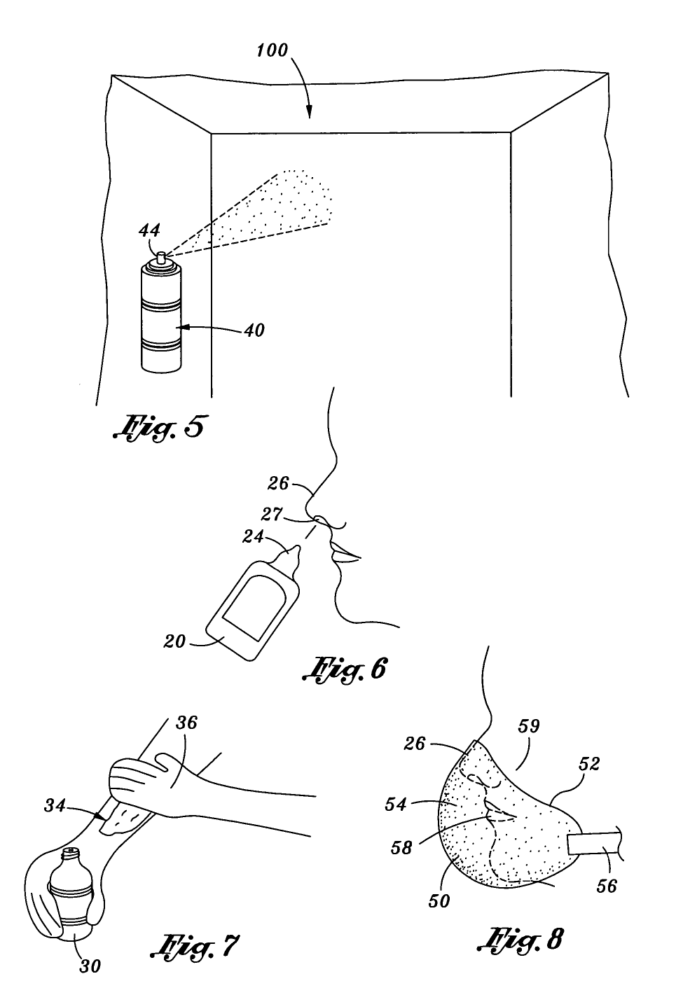

FIG. 6 shows an individual employing the inhaler 20. The user first, removes a cap 22 (FIG. 3A) from the inhaler 20 and then brings the tip 24 of the inhaler 20 up to his or her nose 26 and inserts the tip 24 into one nostril 27. Once the tip 24 has been inserted into the nostril 27, the user squeezes the inhaler 20, thereby ejecting at least some of its contents in the form of a mist, and inhales the mist through the nostril 27 in which tip 24 has been inserted and then the other nostril may also be used to receive the mist. This process may be repeated as many times as the user feels necessary. Once the user is secure that the threat has been properly treated he or she may re-attach the cap 22 to the inhaler 20 and return the inhaler 20 to the cavity 21 of the kit 10a or 10b, or discard the inhaler 20 and its cap 22 if the contents therein have been depleted.

FIG. 7 shows an individual applying the lotion from the lotion receptacle 30 to exposed skin 34. The user first removes a cap 32 (FIG. 3A) of the lotion receptacle 30, and then applies an appropriate amount of the receptacle's lotion contents to the user's skin 34. Then the user using his or her hand rubs the lotion into the skin. The lotion is applied to most, if not the entire, exposed skin areas of the user, or even to the entire body of the user. Once the skin has been properly treated, the user may re-attach the cap 32 and return the lotion receptacle 30 to the cavity 31 of the kit 10a or 10b, or discard the lotion receptacle 30 and its cap 32 if the contents therein have been depleted.

FIG. 8 shows an individual wearing the facemask 50 over his or her mouth 58 and nose 26. The facemask 50 is first positioned to ensure that its facemask border 52 is flush against the skin all the way around the mouth 58 and nose 26. The user then stretches an elastic band 56 of the facemask 50 over his or her head 59 until the band 56 is around the entire head and to hold facemask 50 firmly in place. Once the biological threat has ended and the user feels safe enough to breathe without the aid of filtration, the user removes the facemask 50, stretching the band 56 far enough out to allow it pass over the head 59. Upon removal the facemask 50 may be returned to the cavity 51 of the kit 10a or 10b.

### SCOPE OF THE INVENTION

The above presents a description of the best mode contemplated of carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above which are fully equivalent. Consequently, it is not the intention to limit this invention to the particular embodiments disclosed. On the contrary, the intention is to cover all modifications and alternate constructions coming within the spirit and scope of the invention as generally expressed by the following claims, which particularly point out and distinctly claim the subject matter of the invention:

1. A kit comprising:
a manually actuated aerosol spray container holding a bio-attack protection agent,
a manually actuated inhaler containing a bio-attack protection agent, and
a receptacle of skin lotion including a bio-attack protection agent.

2. The kit of claim 1 including a portable case in which the aerosol container, inhaler, and receptacle are stored.

3. The kit of claim 1 capable of floating on water.

4. The kit of claim 1 including a facemask that while worn filters inhaled air to remove substantially harmful airborne biological substances.

5. The kit of claim 1 where the bio-attack protection agent in the spray container comprises a liquid blend of citrus oils, plant herbs, and seed bitters in a water and food grain alcohol solution.

6. The kit of claim 1 where the bio-attack protection agent in the spray container includes one or more of the following: bee propolis extract, melaleuca alternifolia (tea tree) leaf oil, urea, citrus aurantium amara (bitter orange/petitgrain) oil, citrus grandis (grapefruit) fruit extract.

7. The kit of claim 1 where the bio-attack protection agent in the inhaler comprises a liquid blend of citrus oils, plant herbs, and seed bitters in an aqueous solution.

8. The kit of claim 1 where the bio-attack protection agent in the inhaler includes one or more of the following: citrus grandis (grapefruit) extract, alternifolia (tea tree) leaf oil

9. The kit of claim 1 where the bio-attack protection agent in the skin lotion comprises a liquid blend of citrus oils, plant herbs, and seed bitters in a base of olea europaea (olive) fruit oil.

10. The kit of claim 1 where the bio-attack protection agent in the skin lotion includes one or more of the following: bee propolis extract, urea, alternifolia (tea tree) oil, citrus grandis (grapefruit) seed, and fruit extracts.

11. The kit of claim 1 where a major portion of any bio-attack protection agent is a naturally occurring composition or compositions.

12. A kit comprising:
means for applying to an individual's skin a lotion having a bio-attack protection agent,
means for creating an inhalable mist having a bio-attack protection agent present that is directly breathed in by an individual, and
means for introducing an aerosol having a bio-attack protection agent to the atmosphere.

13. A kit comprising:
a case that creates an enclosure that is opened manually, said case including a liner having
a first cavity holding a manually operable device for applying to an individual's skin a lotion including a bio-attack protection agent,
a second cavity holding a manually operable device that enables a user to create an inhalable mist including a bio-attack protection agent present that is directly breathed in by an individual,
a third cavity holding a manually operable device for spraying into the atmosphere an aerosol including a bio-attack protection agent, and
a forth cavity holding a facemask that filters, when worn, inhaled air to remove harmful airborne biological substances.

14. The kit of claim 14 where said kit case includes a handle.

15. The kit of claim 14 where said kit case includes at least one locking device to keep said kit case in a closed position.

16. A method for countering a biological attack in which a harmful biological substances is dispersed in the atmosphere, said method comprising the steps of
(a) providing a user with a portable kit comprising a manually actuated aerosol spray container holding a bio-attack protection agent, a manually actuated inhaler containing a bio-attack protection agent, and a receptacle of skin lotion including a bio-attack protection agent, and
(b) in an enclosed space substantially isolated from ambient air outside said enclosed space, the user while within said enclosed space
(i) sprays at least some of the bio-attack protection agent in the spray container into the enclosed space,
(ii) inhales at least some of the bio-attack protection agent in the inhaler, and
(iii) applies at least some of the lotion to skin areas.

17. The method of claim 16 where the user covers his or her nose and mouth with a facemask that filters inhaled air to remove airborne biological agents.

18. The method of claim 16 where the biological agents is anthrax and the bio-attack protection agent kills or inhibits the deleterious effects of anthrax.

19. The method of claim 16 where the bio-attack protection agent in the spray container comprises a liquid blend of citrus oils, plant herbs, and seed bitters in a water and food grain alcohol solution.

20. The method of claim 16 where the bio-attack protection agent in the spray container includes one or more of the following: bee propolis extract, melaleuca alternifolia (tea tree) leaf oil, urea, citrus aurantium amara (bitter orange/petitgrain) oil, citrus grandis (grapefruit) fruit extract.

21. The method of claim 16 where the bio-attack protection agent in the inhaler comprises a liquid blend of citrus oils, plant herbs, and seed bitters in an aqueous solution.

22. The method of claim 16 where the bio-attack protection agent in the inhaler includes one or more of the following: citrus grandis (grapefruit) extract, alternifolia (tea tree) leaf oil

23. The method of claim 16 where the bio-attack protection agent in the skin lotion comprises a liquid blend of citrus oils, plant herbs, and seed bitters in a base of olea europaea (olive) fruit oil.

24. The method of claim 16 where the bio-attack protection agent in the skin lotion includes one or more of the following: bee propolis extract, urea, alternifolia (tea tree) oil, citrus grandis (grapefruit) seed, and fruit extracts.

25. The method of claim 16 where in step (b) the steps (i), (ii) and (iii) are conducted in the listed sequence.

27. A method for countering a biological attack in which a harmful biological substance is used, comprising the steps of
identifying what harmful biological substance potentially is to be used in the attack, and
providing one or more bio-attack protection agents in a portable kit that enables a user to travel with the kit and have access to such bio-attack protection agents during an attack.

28. The method of claim 27 where the users travels with the kit in an automotive vehicle.

29. The method of claim 28 where the attack includes introducing into the atmosphere the harmful biological substance and the kit includes a manually actuated aerosol spray container holding a bio-attack protection agent, and, while in an enclosed space within the vehicle that is substantially isolated from ambient air outside the vehicle, the user sprays at least some of the bio-attack protection agent in the spray container into the enclosed space.

30. The method of claim 27 where a user while within an enclosed space, in sequence
(i) sprays at least some of a bio-attack protection agent in a spray container into the enclosed space,
(ii) inhales at least some of a bio-attack protection agent in an inhaler, and
(iii) applies to skin areas at least some of skin lotion including a bio-attack protection agent.
